(19) United States
(12) Patent Application Publication
(10) Pub. No.: US 2013/0102983 A1
(43) Pub. Date: Apr. 25, 2013

(54) SYSTEMS AND METHODS FOR A FLUID-ABSORBING MEMBER

(71) Applicant: Karen Wynne Gilmartin, Boulder, CO (US)

(72) Inventor: Karen Wynne Gilmartin, Boulder, CO (US)

(21) Appl. No.: 13/650,579

(22) Filed: Oct. 12, 2012

Related U.S. Application Data
(60) Provisional application No. 61/546,475, filed on Oct. 12, 2011.

(51) Int. Cl. A61F 13/539; A61F 13/15; A61F 13/14

(52) U.S. Cl. CPC A61F 13/539; A61F 13/141; A61F 13/15617
USPC 604/372; 604/374; 156/93

(57) ABSTRACT

Some embodiments of the invention include a fluid-absorbing member that can include a plurality of layers. In one embodiment, the plurality of layers can include a first layer, a second layer, and a third layer. Further, the first layer can be made from a bamboo-containing material and can be configured to be positioned immediately adjacent to a user's skin. Moreover, the third layer can be made from a fluid-impermeable material and can be configured to be positioned immediately adjacent to the user's clothing. In addition, in some embodiments, the plurality of layers can be coupled together at a position substantially adjacent to an edge of the plurality of layers.

### CROSS-REFERENCE TO RELATED APPLICATIONS

The present application claims priority under 35 U.S.C. §119(e) to U.S. Provisional Application No. 61/546,475 filed on Oct. 12, 2011, the entire contents and disclosure of which are herein incorporated by reference.

### FIELD

This disclosure is related generally to the field of fluid absorbency, and in particular to a system and method for a fluid-absorbent member that is configured and arranged to provide absorbency during lactation.

### BACKGROUND

Breast feeding is a common manner in which a nursing mother provides nourishment to her child. Under some circumstances, milk may involuntarily efflux from one or both of the breasts of the nursing mother, which may be a source of embarrassment and discomfort. For example, during instances commonly known as “let down,” milk may be spontaneously (i.e., without prompting from a feeding child) flow from one or both of the breasts, which may saturate the mother's undergarments and shirt. In other situations, such as nursing a single child, milk may be drawn from a first breast (e.g., the right breast) by the feeding child; however, milk may involuntarily flow from the second breast (e.g., the left breast) in addition to the first breast, which may also create an uncomfortable situation for the nursing mother.

To date, multiple conventional nursing pads have been developed to alleviate some or all of the difficulties associated with involuntary release of milk during lactation. For example, nursing pads may be positioned within a brassiere between one or both of the breasts and the cups of the brassiere. As a result, at least some of these conventional nursing pads can absorb a portion of the involuntarily released milk to minimize the amounts of milk that are absorbed by the mother's clothing. Many of these conventional nursing pads exhibit shortcomings such as lack of comfort for the mother when wearing the nursing pads, bunching of the nursing pads within the brassiere so that it is readily apparent that the mother is wearing the nursing pads, or providing ineffective or non-existent absorbency.

### SUMMARY

Some embodiments of the invention include a fluid-absorbing member, which may include a plurality of layers. In one embodiment, the plurality of layers can include a first layer, a second layer, and a third layer. The first layer may be manufactured from a bamboo-containing material and can be configured to be positioned immediately adjacent to a user's skin. Moreover, in one embodiment, the third layer can be made from a fluid-impermeable material and can be configured to be positioned immediately adjacent to the user's clothing. In addition, in some embodiments, the plurality of layers can be coupled together at a position substantially adjacent to an edge of each of the plurality of layers.

Some further embodiments of the invention include a fluid-absorbing member that includes a plurality of layers. In one embodiment, at least one of the plurality of layers can be substantially or completely impermeable to one or more fluids, such as fluids produced by a human. In addition, in one embodiment, the plurality of layers may be configured to include a first lobe and a second lobe, with the first lobe and the second lobe intersecting at a first vertex. Moreover, the first vertex can be formed so that it defines an angle of less than about 94 degrees. As a result, the fluid-absorbing member can exhibit a substantially heart-shaped configuration.

Some further embodiments of the invention can include a method of assembling a fluid-absorbing member. In some embodiments, the method can include providing a first layer that may comprise a bamboo-containing material and a second layer that may comprise an absorbent material. In some embodiments, the absorbent material may be a hemp-containing material. The method can further include providing a third layer made from a fluid-impermeable material and then coupling together the second layer and the third layer via a lamination process. In some embodiments, the first layer can then be coupled to the second and the third layers.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is a front view of a fluid-absorbing member according to one embodiment of the invention;

FIG. 2 is a front view of the fluid-absorbing member of FIG. 1 that has been modified to illustrate a first layer and a second layer;

FIG. 3 is an exploded view of a fluid-absorbing member according to one embodiment of the invention;

FIG. 4 is a front view of the fluid-absorbing member of FIG. 1;

FIG. 5 is a front view a woman using the fluid-absorbing member of FIG. 1;

FIG. 6A is a series of side views of the fluid-absorbing member of FIG. 1;

FIG. 6B is a series of side views of a fluid-absorbing member according to another embodiment of the invention; and

FIG. 7 is an exploded view of the fluid-absorbing member of FIG. 5B.

Corresponding reference characters indicate corresponding elements among the view of the drawings. The headings used in the figures should not be interpreted to limit the scope of the claims.

### DETAILED DESCRIPTION

The following description can be generally applicable to an improved heart-shaped nursing pad. Specifically, the improved heart-shaped nursing pad exhibits improved absorbency and heat retention so that the wearer does not experience the cold, wet feeling often associated with many of the nursing pads currently on the market. At least in part, these improvements arise as a result of the three-layered design that includes two absorbent layers and a single layer that prevents the absorbed liquid from being transferred to the wearer's clothes. Moreover, at least one of the two absorbent layers is made of a bamboo fiber, which provides a soft feel to the improved nursing pad so that the wearer's skin is not irritated during use. In addition, the heart shape enables the improved nursing pad to readily contour itself to the body of any nursing mother so that the pad does not readily wrinkle and appear visible through the clothing.

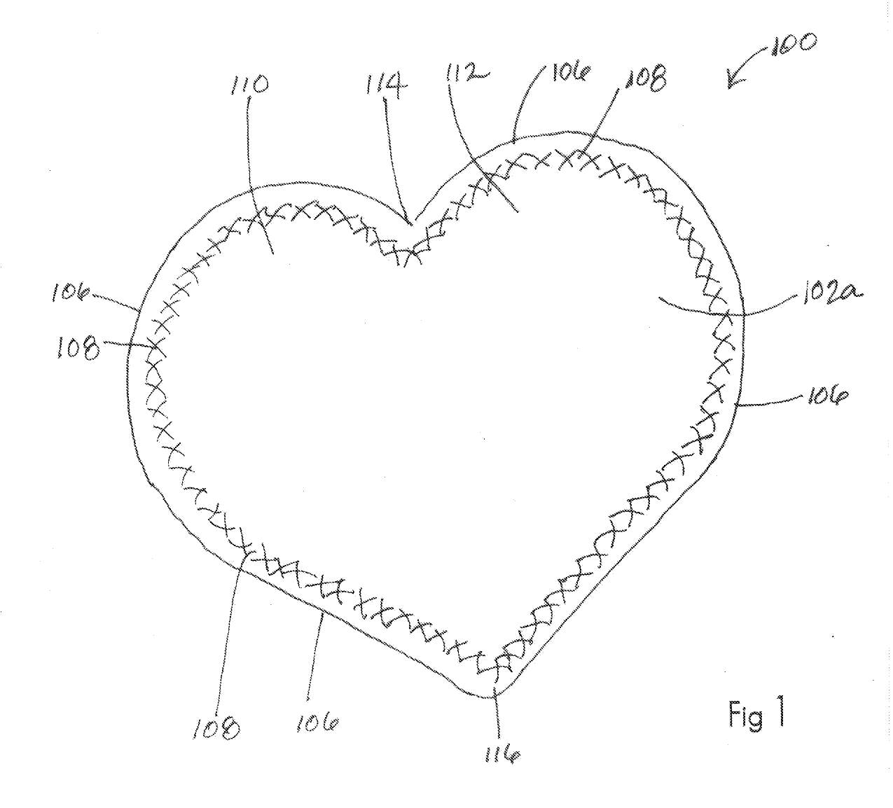

Referring to the drawings, embodiments of a fluid-absorbing member is illustrated and generally indicated as 100 in FIGS. 1-5A. In one embodiment, the fluid-absorbing member 100 may be used by one or more lactating women to at least partially aid against the involuntary release of milk. By way of example only, in some embodiments, a fluid-absorbing member 100 can be placed between a brassiere and the breasts of the lactating mother. In particular, a first fluid-absorbing member 100 may be placed between a left breast and a first cup of the brassiere and a second fluid-absorbing member 100 may be placed between a right breast and a second cup of the brassiere. As described in further detail below, the first and the second fluid-absorbing members 100 can be configured and arranged to absorb at least a portion of the milk that involuntarily effluxes from one or both breasts. As a result, at least some of the milk that would have been absorbed by clothing worn by the nursing mother is absorbed by one or both of the fluid-absorbing members 100. Moreover, the following disclose describes the fluid-absorbing member 100 as potentially used by nursing mothers. However, this disclosure is not intended to limit the potential uses of the fluid-absorbing member 100 for use only by nursing mothers. Embodiments of the fluid-absorbing member 100 can be employed in any circumstance in which fluid absorption may be desired and/or necessary.

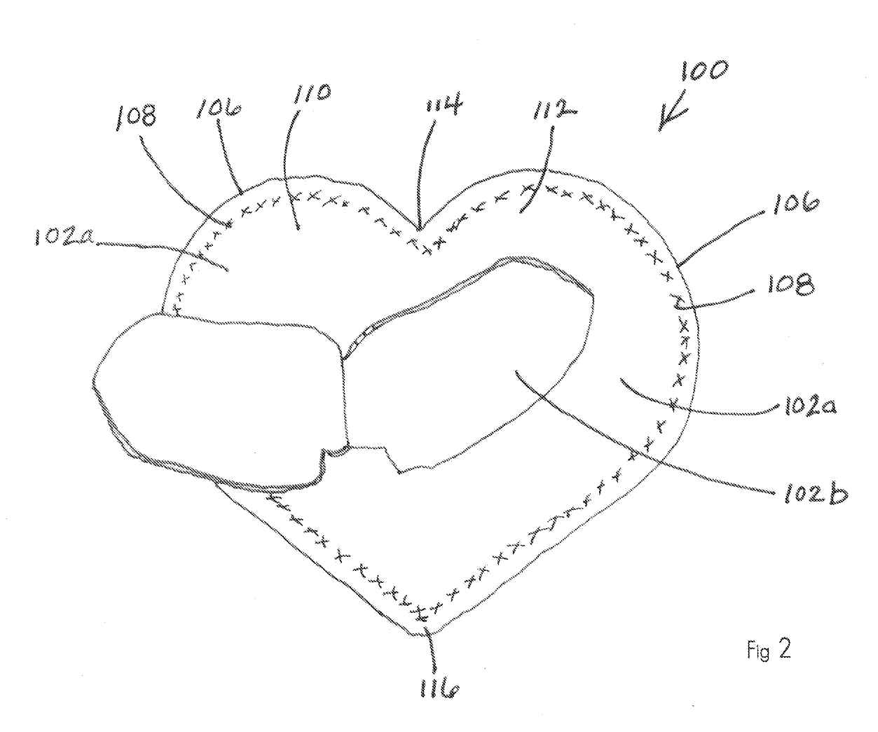

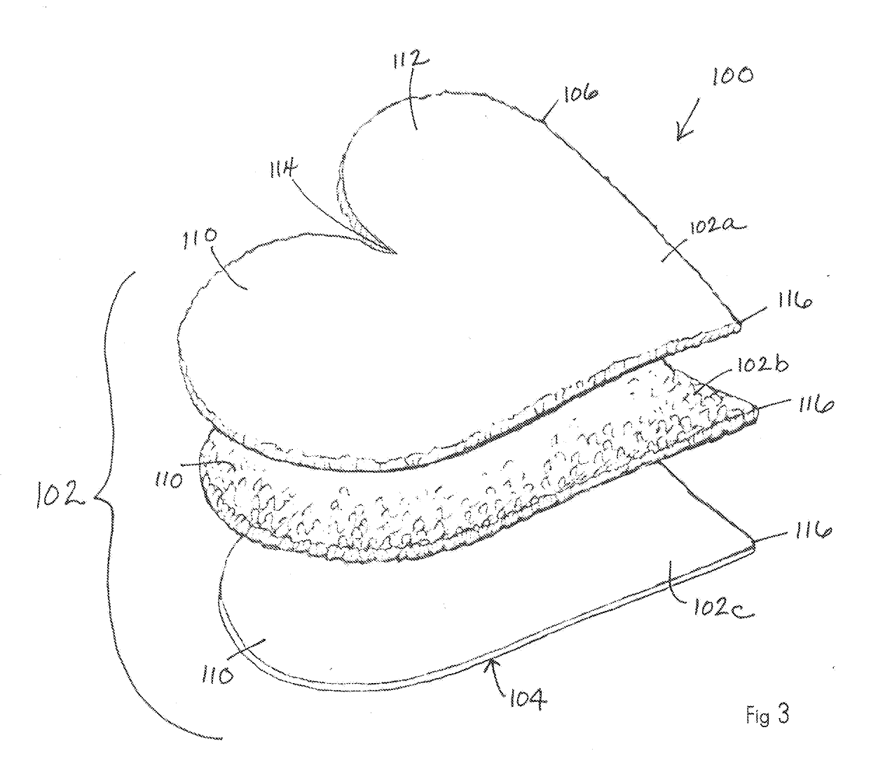

As shown in FIGS. 2 and 3, in some embodiments, the fluid-absorbing member 100 can comprise at least one layer 102. Fox example, the fluid-absorbing member 100 can be made from a plurality of layers 102. In particular, in some embodiments, the plurality of layers 102 can include a first layer 102a, a second layer 102b, and a third layer 102c, as shown in FIG. 3. Specifically, as described in greater detail below, the plurality of layers 102 can be coupled together to form fluid-absorbing member 100. In other embodiments, the fluid-absorbing member 100 can include greater or lesser numbers of layers 102 to suit manufacturer and/or end user needs or requirements.

In some embodiments, the first layer 102a can be positioned within the plurality of layers 102 so that when the fluid-absorbing member 100 positioned adjacent to a breast, the first layer 102a is in contact with the breast. In one embodiment, the first layer 102a can be fabricated from a soft, cushioned, bendable, flexible, or otherwise comfortable material so that when fluid-absorbing member 100 is immediately adjacent to the breast, it does not irritate the breast, the nipple, or other portions of the areola. For example, the first layer 102a can be configured to provide succor to some areas of the breast (e.g., the nipple) that may be irritated, damaged, or otherwise experiencing discomfort as a result of the nursing process.

In some embodiments, the first layer 102a can be fabricated from a material comprising one or more natural fibers (e.g., organically certified fibers) and/or one or more man-made fibers. For example, the first layer 102a can be made from the fibers of cotton, silk, bamboo, hemp, polyester, or any other suitable fibers. In one embodiment, the first layer 102a can be manufactured from a composition that includes more than one of the above-mentioned materials. In particular, the first layer 102a can be manufactured from bamboo, cotton, and/or. polyester (e.g., a bamboo rayon and cotton velour). By way of example only, in one embodiment, the first layer 102a can be manufactured from about 78% bamboo rayon, about 28% organic cotton, and about 2% polyester. In other embodiments, the first layer 102a can be made from any other combination of the natural and man-made fibers, including any compositions that include bamboo as a constituent. Moreover, in some embodiments, the first layer 102a can comprise any suitable woven and non-woven fabrics.

The inclusion of bamboo as a constituent of the first layer 102a can provide benefits to the fluid-absorbing member 100 that are not present in some conventional nursing pads. In particular, the fibers that make up bamboo comprise a generally round, cylindrical, or circular cross-section so that the fibers themselves do not irritate the breast tissue, including the nipple. As a result, the fluid-absorbing member 100 that includes a first layer 102a comprising bamboo fibers can be less irritating to nursing mother relative to conventional nursing pads, which are generally made from materials such as cotton and polyester (i.e., non-bamboo materials), which may more readily irritate the breast. Moreover, because of the rounded nature of the bamboo fibers, the first layer 102a may sit closer to the skin of the breast relative to other materials without rounded fibers (e.g., cotton or polyester). For example, when spun into yarn, bamboo fibers can exhibit a softness that is similar to silk fiber, thereby making the use of the fluid-absorbing member 100 more pleasant for the nursing mother.

In addition, the inclusion of bamboo as a constituent of the first layer 102a may also offer additional benefits. First, bamboo fiber can be more absorbent than other materials, such as cotton. More specifically, the natural configuration of bamboo fibers includes multiple microgaps and microholes (not shown) that can more readily receive fluids (e.g., involuntarily released milk) relative to other materials, such as cotton. For example, bamboo materials can be up to four times as absorbent as cotton materials of a similar size. Moreover, the microgaps and microholes provide an enhanced capacity for ventilation of the area immediately adjacent to the first layer 102a. As a result, when the fluid-absorbing member 100 is positioned adjacent to the breast, relative to a conventional, all-cotton nursing pad, the fluid-absorbing member 100 can absorb greater amounts of milk, which can result in a reduction (e.g., a complete or partial reduction) in the amount of milk that may be absorbed by the clothing of the nursing mother. Furthermore, the increased capacity for ventilation can also enable improved evaporation of the absorbed fluids, relative to an all-cotton nursing pad. In addition to absorbing involuntarily released milk, the fluid-absorbing member 100 may also readily absorb sweat and other fluids, which may provide further comfort for the nursing mother.

In addition, bamboo fibers can more readily retain thermal energy, relative to other fibers. As a result, the natural thermal energy radiated by the human body can be received and retained by the first layer 102a, which may provide a more pleasant warming sensation for the nursing mother relative to conventional nursing pads that comprise materials (e.g., cotton-only materials) that do not retain thermal energy to the same extent and may result in a “cold and clammy” sensation when wet. Accordingly, a first layer 102a formed from a material that includes bamboo can provide a more pleasant experience for the nursing mother by exhibiting a softer feel on the skin, which may or may not already be irritated due to the nursing process, and improved absorbability and thermal energy retention relative to all-cotton nursing pads.

In some embodiments, the second layer 102b may also be manufactured from an absorbent material. Particularly, the second layer 102b can comprise at least one of the previously mentioned materials from which the first layer 102a may be manufactured. In one embodiment, the second layer 102b may be manufactured from a material comprising hemp and other natural and/or man-made woven or unwoven fibers. By way of example only, the second layer 102b can comprise about 55% hemp and about 45% organic cotton. The inclusion of hemp can further improve the absorbency and thermal energy retention of the fluid-absorbing member 100. Specifically, similar to bamboo, hemp can also be more absorbent than cotton (i.e., at least four times more absorbent than cotton) and hemp can also retain greater amounts of naturally rejected body heat. As a result, the fluid-absorbing member 100 can retain additional amounts of fluids (e.g., milk) and body heat to provide a more pleasant experience for the nursing mother.

In some embodiments, the third layer 102c may be manufactured from a material with one or more dissimilar properties relative to the materials of the first and second layers 102a, 102b. Specifically, in one embodiment, the third layer 102c can be configured as partially or completely impermeable to fluids. For example, in some embodiments, the third layer 102c may comprise a man-made fabric that has been impregnated (e.g., laminated) with a material to improve its impermeability to one or more fluids. By way of example only, in one embodiment, the third layer 102c can comprise polyester that has been impregnated with a fluid-proofing agent, such as polyurethane and/or a bio-plastic-based compound. The impregnation and/or lamination process can be performed so that the polyester material is covered by a thin film of the fluid-proofing agent or an extruded liquid polymer can be applied to the polyester layer. In addition, any fibrous material (e.g., woven or unwoven) can be used in addition to or in lieu of polyester. Moreover, in some embodiments, the third layer 102c may also comprise materials similar to those used in manufacture of the first and second layers 102a, 102b.

As a result of the fluid-proofing process of the third layer 102c, the fluid-absorbing member 100 can offer advantages relative to conventional, all-cotton nursing pads. For example, when assembled and used to absorb involuntarily released milk, the fluid-absorbing member 100 can absorb some or all of the involuntarily released milk via the first and second layers 102a, 102b, and the third layer 102c can prevent the transfer of some or all of the milk to the brassiere and/or clothing. Many conventional all-cotton nursing pads will not prevent the transfer of the absorbed milk from the pads to the clothing of the nursing mother, which can embarrass the nursing mother and make her feel uncomfortable due to the wetness of her clothing.

Moreover, the third layer 102c can include an aesthetically pleasing color or surface pattern to suit the desires of the nursing mother. For example, an outer surface 104 of the third layer 102c can be outward facing when the fluid-absorbing member 100 is placed between the breast and the cup(s) of the brassiere. Accordingly, should a nursing mother desire a certain pattern, design, or color, the third layer 102c can comprise this pattern, design, or color to meet the aesthetic needs of the nursing mother.

In some embodiments, the plurality of layers 102 can be assembled to form the fluid-absorbing member 100. For example, in one embodiment, the second and third layers 102b, 102c can be coupled together prior to attachment of the first layer 102a. By way of example only, the second and third layers 102b, 102c can coupled together using a lamination process. In one embodiment, the lamination process can be substantially similar to the lamination process described above with respect to the third layer 102c. In other embodiments, the lamination process can be any other suitable conventional lamination process. As a result, the second and third layers 102b, 102c can be temporarily or permanently coupled together as a single element. In yet other embodiments, the second and third layers 102b, 102c can be coupled together using any other conventional coupling techniques such as mechanical attachment (e.g., sewing, stapling, etc.) or can be left uncoupled so that the plurality of layers 102 are assembled together at substantially the same time.

In some embodiments, by laminating the third layer 102c and laminating together the second and third layers 102b, 102c, the fluid-absorbing member 100 can exhibit improved properties relative to a non-laminated conventional nursing pad. For example, the lamination process can confer a structural stability on the second and third layers 102b, 102c. In particular, many conventional nursing pads comprise an all-cotton configuration that may readily wrinkle when placed between the breast and the brassiere, which may result in the nursing pad being visible through the clothing. However, after the lamination process, the laminated second and third layers 102b, 102c may provide additional structural stability so that the fluid-absorbing member 100 does not substantially wrinkle, thereby reducing the chances that the fluid-absorbing member 100 will be visible through the clothing. However, it should be noted that the second and third layers 102b, 102c may only be lightly laminated. As a result, the lamination can provide structural stability and a general capability to prevent milk from transferring to the brassiere and clothing, but the fluid-absorbing member 100 can still retain sufficient flexibility so that the nursing mother can fold, bend, or otherwise reconfigure the fluid-absorbing member 100 during nursing so that there is not a need to remove the fluid-absorbing member 100 when feeding a child.

In addition, the lamination process can reduce or eliminate the chances of bubbling and the separation of the plurality of layers 102 from occurring. Moreover, the lamination of at least some of the plurality of layers 102 enables the use of materials with a relatively lesser thickness to be used in the construction of the fluid-absorbing member 100. As a result of using less thick materials, the fluid-absorbing member 100 is less likely to be visible through the brassiere and/or clothing worn by the nursing mother.

In some embodiments, after coupling together the second and third layers 102b, 102c, the first layer 102a can be added to form the fluid-absorbing member 100. In particular, the first layer 102a can be positioned so that the second layer 102b is disposed between the first and third layers 102a, 102c. As a result, the plurality of layers 102 can be positioned so that, when positioned between the breast and the brassiere, the first layer 102a is immediately adjacent to the breast and the third layer 102c is immediately adjacent to the brassiere.

In some embodiments, once the plurality of layers 102 are substantially or completely aligned, the first layer 102a can be coupled to the second and third layers 102b, 102c. In some embodiments, one or more mechanical coupling techniques can be used to couple together the plurality of layers 102. For example, in one embodiment, the plurality of layers 102 can be sewn together. As can be seen in FIGS. 1 and 2, the plurality of layers 102 can be sewn together at a position substantially adjacent to an edge or periphery 106 of the fluid-absorbing member 100. Specifically, stitching 108 can be introduced adjacent to the periphery 106 and can extend around some or all of the perimeter of the fluid-absorbing member 100. In one embodiment, the stitching 106 may extend through some or all of the plurality of layers 102 to retain together the layers 102 of the fluid-absorbing member 100.

In some embodiments, different sewing and stitching techniques can be used to form the fluid-absorbing member 100. For example, as shown in FIGS. 1, 2, and 4, a conventional “zigzag” sewing stitch can be used to couple together the plurality of layers 102. The zigzag sewing stitch can be more comfortable against the skin of the nursing mother relative to sewing stitches used in some conventional nursing pads. Specifically, some conventional nursing pads employ other stitched configurations (e.g., a “serge” stitched edge), which may create an unpleasant and/or uncomfortable texture on the skin of the nursing mother. For instance, a serge-stitched edge produces a thick and bulky periphery, which may be unpleasant to the touch and may also result in the conventional nursing pad being visible through the clothing. Because the zigzag sewing stitch produces a generally less thick and bulky periphery 106, the fluid-absorbing member 100 can be more comfortable for nursing mothers relative to conventional nursing pads.

In addition, by providing the stitching 106 substantially adjacent to the periphery 108, stitching 106 in the areas of the fluid-absorbing member 100 that may be adjacent to sensitive areas of the nursing mother can be avoided. Some conventional nursing pads may have one or more seams along the surface of the pad that contacts the nipple or areola, which may be sensitive and/or irritated due to the nursing process. These seams may further irritate this already-sensitive region of the nursing mother. By providing the stitching 106 at the periphery 108 of the fluid-absorbent member 100, contact with some of the more-sensitive areas of the nursing mother can be avoided, thereby producing a more pleasant experience for the mother.

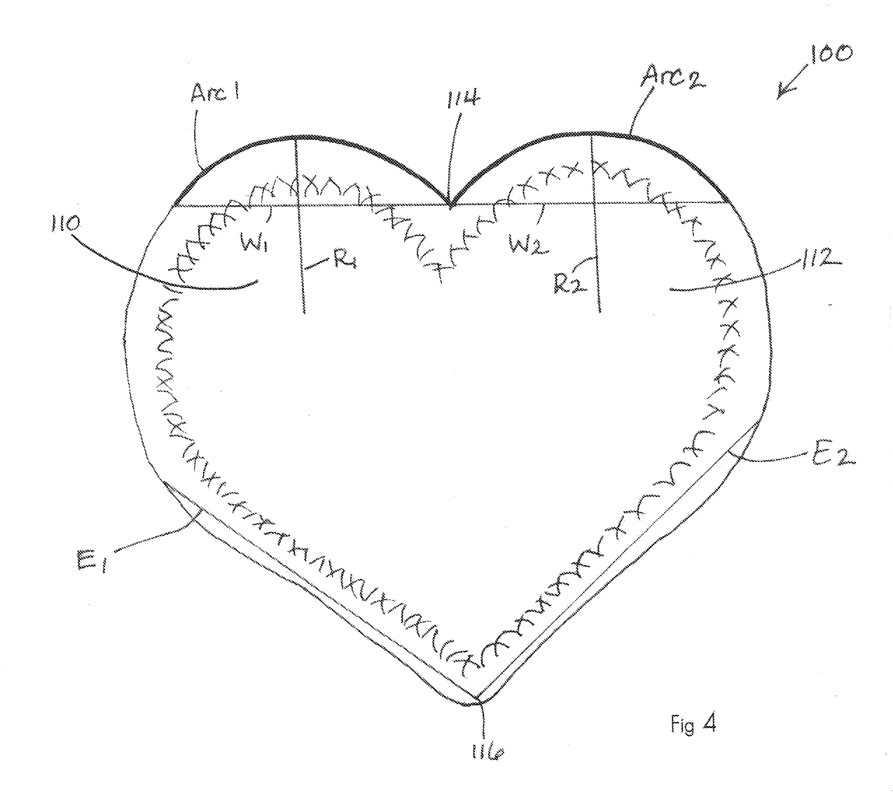

As shown in FIGS. 1-5, the fluid-absorbing member 100 may comprise a generally heart-shaped configuration. Specifically, the fluid-absorbing member 100 can include a first lobe 110, a second lobe 112, a first vertex 114, and a second vertex 116. As best viewed in FIGS. 1 and 4, the first vertex 114 can be positioned generally between the first and the second lobes 110, 112. In addition, the second vertex 116 can be positioned near a bottom portion of the fluid-absorbing member 100 and substantially aligned with the first vertex 114. As a result of this configuration, in some embodiments, the fluid-absorbing member 100 exhibits a heart-shaped configuration. In some embodiments, some or all of the plurality of layers 102 can be formed in a heart-shaped configuration prior to being assembled to form the fluid-absorbing member 100. However, in other embodiments, the fluid-absorbing member 100 can be reconfigured into the heart-shaped configuration after coupling of the plurality of layers 102.

In some embodiments, the first and the second lobes 110, 112 can be sized to comfortably fit some or all nursing mothers. As shown in FIGS. 1-4, in one embodiment, the first and second lobes 110, 112 can exhibit an at least partially curved, arched, semi-circular, or otherwise rounded configuration with the first vertex 114 being defined therebetween. More specifically, the first and second lobes 110, 112 may define substantially arcuate contours that intersect at an angle that defines the first vertex 114. Similarly, at the lower portion of the fluid-absorbing member 100, the edges 106 can similarly intersect at an angle to define the second vertex 116.

For example, referring now to FIG. 4, the first and the second lobes 110, 112 can include a first arc Arc1 and a second arc Arc2, respectively. In some embodiments, as shown in FIG. 4, the first and second arcs Arc1, Arc2 can comprise substantially similar sizes and configurations. For example, in one embodiment, the arcs Arc1, Arc2 may each be about 2.125 inches wide (W1 and W2), have an arc radius of about 1.38 inches (R1 and R2), and an arc length of about 2.43 inches. In one embodiment, the first vertex 114 can be formed at the intersection of the first and second arcs Arc1, Arc2, and, because of the configuration mentioned above, the first vertex 114 can comprise an angle of about 94 degrees or less. For example, the first vertex 114 can comprise an angle within the range of about 94 degrees to about 80 degrees, preferably in the range of about 92 degrees to about 86 degrees. By way of example only, the first vertex 114 may be about 90 degrees. In other embodiments, these dimensions can vary to meet end user or manufacturer needs or requirements, and the size and angles of the first vertex 114 can accordingly vary. Moreover, in some embodiments, the constituent elements of the first and second lobes 110, 112 can comprise different sizes, relative to each other (i.e., the first arc Arc1 comprises different dimensions relative to the second arc Arc2).

In addition, as previously mentioned, the second vertex 116 can be formed at an intersection of the edges 106 of the fluid-absorbing member 100. In particular, as shown in FIG. 4, the fluid-absorbing member 100 can include edges 106 that extend from the first and second lobes 110, 112 to the second vertex 116. For example, a first edge E1 can extend from the first lobe 110 toward the second vertex 116 and a second edge E2 can extend from the second lobe 112 toward the second vertex 116. In some embodiments, the first and second edges E1, E2 can comprise a length of about 3 inches. In some embodiments, the edges E1, E2 can meet at the second vertex 116 so that the second vertex 116 comprises an angle of about 85 degrees or greater. For example, the second vertex 116 can comprise an angle within the range of about 105 degrees to about 85 degrees, preferably in the range of about 98 degrees to about 90 degrees. By way of example only, the second vertex 116 may be about 90degrees. In other embodiments, the edges E1, E2 can comprise any other length to meet end user or manufacturer needs or requirements.

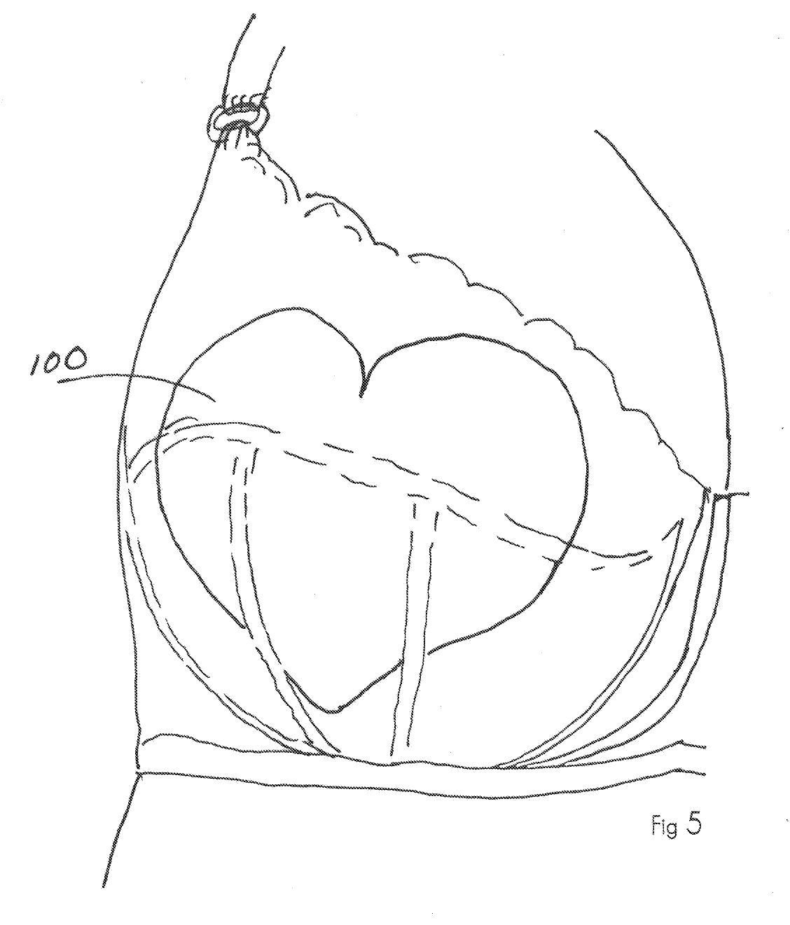

Moreover, in some embodiments, the vertices 114, 116 can be configured to enable the fluid-absorbing member 100 to dynamically change shape to match the contours of the nursing mother. For example, as previously mentioned, the first and/or the second vertices 114, 116 may comprise an angle of about 90 degrees. In one embodiment, both the first and the second vertices 114, 116 include angels of about 90 degrees. As a result of the first and second vertices 114, 116 comprising about 90 degree angles, the fluid-absorbing member 100 is capable of providing three-dimensional contouring to the breasts of the nursing mother without wrinkling, as shown in FIG. 5. More specifically, the first and the second vertices 114, 116 enable the first and the second lobes 110, 112 to be contoured to the size and shape of the breast and to further allow the lobes 110, 112 to move largely independent of each other, which enables the fluid-absorbing member 100 to be closely formed to the breast, as illustrated in FIG. 5. As a result of being able to be closely formed to the breast, the fluid-absorbing member 100 may resist wrinkling, which can reduce or eliminate visibility through the clothing of the nursing mother. In other embodiments, the first and/or the second vertices 114, 116 can comprise other angles desired by manufacturers and/or end users.

In addition, by being able to dynamically change shape, the fluid-absorbing member 100 can remain in close contact with the breast no matter the shape and/or size. More specifically, the size and shape of the breasts of a nursing mother routinely change due to the production and subsequent release of milk (i.e., either through involuntary release or feeding of the child). Accordingly, by being able to dynamically configure itself to the changing size and shape of the nursing mother, the fluid-absorbing member 100 can constantly remain in contact with the breast to provide comfort and absorption.

As a result of this configuration, the fluid-absorbing member 100 can exhibit improved comfort and absorbency relative to conventional nursing pads. Specifically, the plurality of layers 102 can provide improved absorbency and thermal energy retention because the use of bamboo and hemp in the first and second layers 102a, 102b, respectively. Moreover, by including a laminated or otherwise fluid impermeable third layer 102c, the fluids (e.g., involuntarily released milk) absorbed by the first and second layers 102a, 102b can be retained within the fluid-absorbing member 100 until cleaning. Furthermore, fluid-absorbing member 100 can be more comfortable for the nursing mother because the first layer 102a comprises bamboo, which is a less irritating material relative to all-cotton nursing pads. In addition, by forming the fluid-absorbing member 100 in a heart-shaped configuration with a first and second vertex 114, 116 comprising ninety degree angles, the fluid-absorbing member 100 may closely contour the dynamic sizing of the nursing mother. In addition, because of the fiber-based construction, the fluid-absorbing member 100 can be repeatedly cleaned and reused during the nursing process.

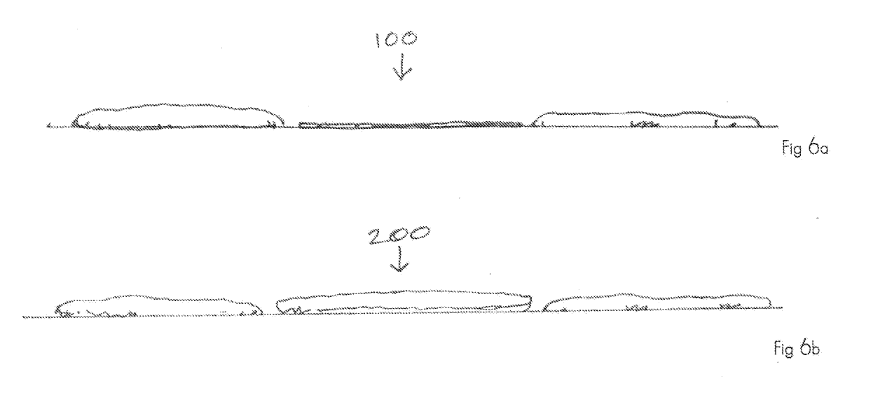

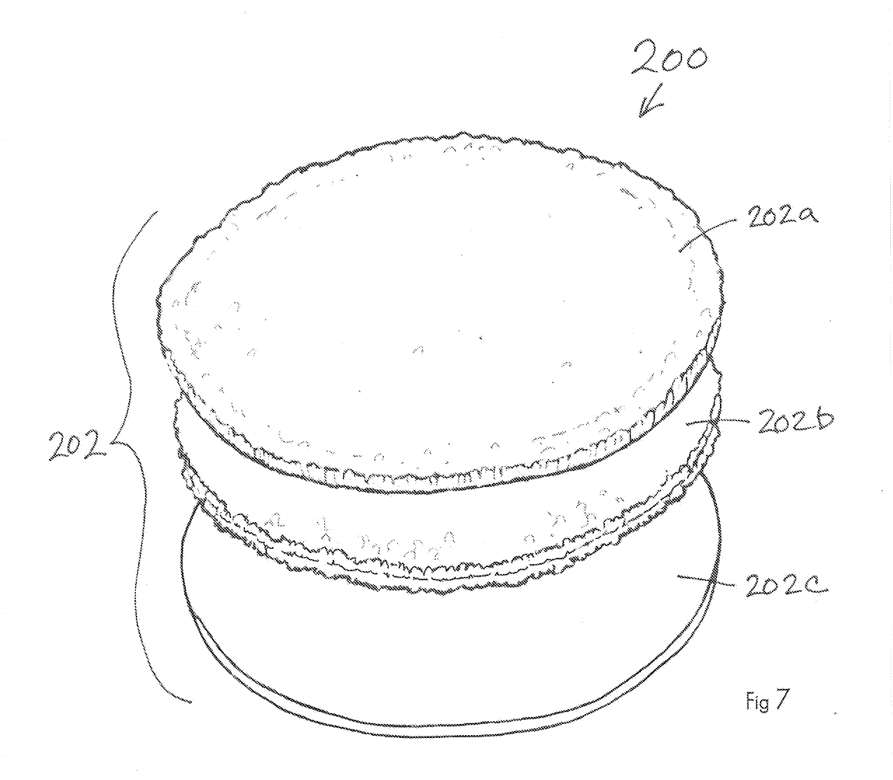

FIGS. 6B and 7 illustrate another embodiment of the fluid-absorbing member 200. In some embodiments, the fluid-absorbing member 200 can exhibit a different configuration for different requirements of the nursing mother. As shown in FIGS. 5A and 5B, the fluid-absorbing member 200 can comprise a relatively thicker and more absorbent configuration, relative to other embodiments of the invention. As a result, the fluid-absorbing member 200 can be well suited for use over longer periods of time. In addition, the fluid-absorbing member 200 may be well suited for use in absorbing greater amounts (i.e., volumes) of milk. By way of example only, the fluid-absorbing member 200 can be used at night while the nursing mother is sleeping. Specifically, during overnight hours, many nursing mothers will feed their child less than during daytime hours which leads to greater amounts of milk accumulating, thereby leading to greater amounts of involuntarily released milk. By providing a thicker fluid-absorbing member 200, greater amounts of milk can be absorbed.

In addition, in some embodiments, the fluid-absorbing member 200 can exhibit a different shape. For example, as shown in FIG. 7, the fluid-absorbing member 200 can comprise a substantially circular or cylindrical configuration. Although this configuration may result in visibility through the clothing of the nursing mother, because the fluid-absorbing member 200 is generally worn during the night, the nursing mother may have less concern over the potential wrinkles and visibility associated with fluid-absorbing member 200.

In some embodiments, the fluid-absorbing member 200 may also comprise a plurality of layers 202, as shown in FIG. 7. At least some of the plurality of layers 202 can comprise materials similar to those used in forming the plurality of layers 102. For example, in some embodiments, the first layer 202a may comprise a bamboo-containing material and the second layer 202b can comprise material that includes a mixture of hemp and cotton. As a result, the fluid-absorbing member 200 can exhibit the same comfort and absorbency that was previously mentioned with respect to previous embodiments. In some embodiments, the third layer 202c can be configured in a manner substantially similar to the first layer 202a. In some embodiments, the first layer 202a and the third layer 202c can be substantially or completely identical. In particular, the first and third layers 202a, 202c may each comprise a bamboo-containing material so that should a first side of the fluid-absorbing member 200 become totally or partially saturated with milk (e.g., the first layer 202a), the nursing mother can rearrange the fluid-absorbing member 200 so that the third layer 202c is immediately adjacent to the breast to absorb another volume of milk (i.e., flip over the fluid-absorbing member 200). In other embodiment, the third layer 202c can comprise a substantially fluid-impermeable configuration, similar to some previously mentioned embodiments.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A fluid-absorbing member comprising:
a plurality of layers including a first layer, a second layer, and a third layer, wherein
the first layer comprises a bamboo-containing material and is configured to be positioned immediately adjacent to a user's skin,
the third layer comprises a fluid-impermeable material and is configured to be positioned immediately adjacent to the user's clothing, and
the plurality of layers are coupled together at a position substantially adjacent to an edge of each of the plurality of layers.

2. The fluid-absorbing member of claim 1, wherein the plurality of layers are sewn together.

3. The fluid-absorbing member of claim 2, wherein the plurality of layers are sewn together using a zig-zag sewing stitch.

4. The fluid-absorbing member of claim 1, wherein the second layer comprises a hemp-containing material.

5. The fluid-absorbing member of claim 1, wherein the second layer is disposed between the first layer and the third layer.

6. The fluid-absorbing member of claim 5, wherein the first layer and the second layer are configured to absorb a fluid.

7. The fluid-absorbing member of claim 6, wherein the fluid is one of milk and perspiration.

8. The fluid-absorbing member of claim 1, wherein the plurality of layers comprise a heart-shaped configuration.

9. The fluid-absorbing member of claim 1, wherein the third layer comprises polyurethane laminated polyester.

10. The fluid-absorbing member of claim 1, wherein the second layer and the third layer are coupled together using a lamination process.

11. A fluid-absorbing member comprising:
a plurality of layers, wherein at least one of the plurality of layers is substantially impermeable to one or more fluids; and
the plurality of layers being configured to include a first lobe and a second lobe, wherein the first lobe and the second lobe intersect at a first vertex, and wherein the first vertex comprises an angle of less than about 94 degrees.

12. The fluid-absorbing member of claim 11 and further comprising a second vertex.

13. The fluid-absorbing member of claim 11, wherein the plurality of layers comprises a first layer, a second layer, and third layer, and wherein the third layer is substantially impermeable to one or more fluids and is laminated.

14. The fluid-absorbing member of claim 13, wherein the first layer comprises a bamboo-containing material.

15. The fluid-absorbing member of claim 11, wherein the first lobe and the second lobe comprise a first arc and a second arc, and wherein at least one of the first arc and the second arc comprises an arc radius of about 1.38 inches.

16. The fluid-absorbing member of claim 11, wherein the plurality of layers comprise a heart-shaped configuration.

17. The fluid-absorbing member of claim 11 and further comprising a first edge and a second edge, wherein at least one of the first edge and the second edge comprises a length of about 3 inches.

18. A method of assembling a fluid-absorbing member comprising:
providing a first layer comprising a bamboo-containing material;
providing a second layer comprising an absorbent material;
providing a third layer comprising a fluid-impermeable material;
coupling together the second layer and the third layer; and
coupling the first layer to the second layer and the third layer.

19. The method of claim 18, wherein the third layer comprises polyurethane laminated polyester.

20. The method of claim 18, wherein the second layer and the third layer are coupled together using a lamination process and wherein the first layer is coupled to the laminated second layer and third layer using a sewing process.
